(19) United States
(12) Patent Application Publication
(10) Pub. No.: US 2021/0148915 A9
(43) Pub. Date: May 20, 2021

(54) RECOVERY OF ASPARTYL (ASPARAGINYL) BETA HYDROXYLASE (HAAH) FROM AN EXOSOMAL FRACTION OF HUMAN SERA FROM CANCER PATIENTS

(71) Applicant: Panacea Pharmaceuticals, Inc., Gaithersburg, MD (US)

(72) Inventors: Mark Semenuk, Gaithersburg, MD (US); Hossein Ghanbari, Potomac, MD (US); Michael Lebowitz, Baltimore, MD (US)

(21) Appl. No.: 15/828,744

(22) Filed: Dec. 1, 2017

Related U.S. Application Data
(63) Continuation of application No. 14/853,254, filed on Sep. 14, 2015, now abandoned.
(60) Provisional application No. 62/049,582, filed on Sep. 12, 2014.

(51) Int. Cl. G01N 33/574

(52) U.S. Cl. CPC G01N 33/57488; G01N 2333/90245

(57) ABSTRACT

The present invention encompasses methods of detecting exosomes comprising Aspartyl-[Asparaginyl]-β-hydroxylase (HAAH). The present invention contemplates is further directed to methods diagnosing cancer by identifying exosomes comprising HAAH.

### BACKGROUND OF THE INVENTION

Cancer being one of the most devastating diseases both in terms of human life opportunity loss and health care cost, also happens to present highly unmet diagnostic needs. In pursuit of a better understanding of serum from cancer patients as a diagnostic test article, the study of exosomes has greatly emerged. Exosomes are microvesicles of a size ranging between 30-120 nm which are actively secreted through an exocytosis pathway. Exosomes can be secreted under specific physiological conditions from various cell types such as dendritic cells (DC), lymphocytes, mast cells, epithelial cells, and tissue derived from lung, liver, breast, prostate, and colon. Exosomes ultimately appear in the blood and provide an ideal analytical target. Furthermore exosomes may be recovered from d cell culture supernatants and most body fluids, following multistep ultracentrifugation and or polymer induced precipitation processes known in the art. Still further, exosomes inherently carry numerous cancer associated biomarkers and thereby offer valuable non-invasive diagnostic potential.

Aspartyl-(Asparaginyl)-β-hydroxylase (HAAH) is over expressed in various malignant neoplasms, including hepatocellular and lung carcinomas. HAAH is a tumor specific antigen, which is specifically expressed on the surface of certain malignant cells. HAAH is an iron and α ketogluterate dependent hydroxylase enzyme that modifies cellular proteins such as Notch that in turn contribute to cancer etiology by means of causing cell proliferation, motility, and invasiveness. Neutralizing the enzyme or reducing its expression leads to normal phenotype(s) in cancer cells. Anti-HAAH antibodies (as well as siRNA) have been shown to be cytostatic. An all-human sequence anti-HAAH (PAN-622) has shown to inhibit tumor growth by more than 90% in animal studies by passive immunotherapy. However, HAAH is well conserved and is also over expressed in placenta, hence it is not sufficiently immunogenic in animals and it is certainly a self-antigen in humans.

The role of tumor exosomes in cancer progression is an emerging area of study. Given the increasing understanding of the role of exosomes in cancer progression and the fact that there is an increasing need to improve diagnostics methods, there is accordingly a need for methods to detect exosomes comprising tumor specific antigens.

### SUMMARY OF THE INVENTION

The present invention encompasses methods of detecting exosomes comprising Aspartyl-[Asparaginyl]-β-hydroxylase (HAAH).

The present invention further contemplates a method for diagnosing cancer comprising the steps of isolating exosomes from a biological sample, analyzing the exosomes for the presence of HAAH, and diagnosing cancer based on the presence of exosomes comprising HAAH.

Exosomes in accordance with the present invention may by isolated by any means known in the art, including, but not limited to ultracentrifugation or through the use of commercially available kits such as ExoQuick®.

In accordance with the present invention, exosomes may be analyzed by means of ELISA, including, but not limited to HAAH selective analytical sandwich ELISA.

In certain embodiments of the present invention, exosomes are further analyzed for the presence of tissue of origin specific markers in order to determine the type of the diagnosed cancer. Such markers include, but not limited to, markers such as α fetoprotein, CA125, CYFRA 21-1, CEA, and PSA.

The present invention also encompasses methods of recovering HAAH from biological samples.

Further embodiments of the present invention encompass methods of increasing the concentration of HAAH in a biological sample.

### BRIEF DESCRIPTION OF THE FIGURES

FIG. 1 depicts the formation of an HAAH containing exosome.

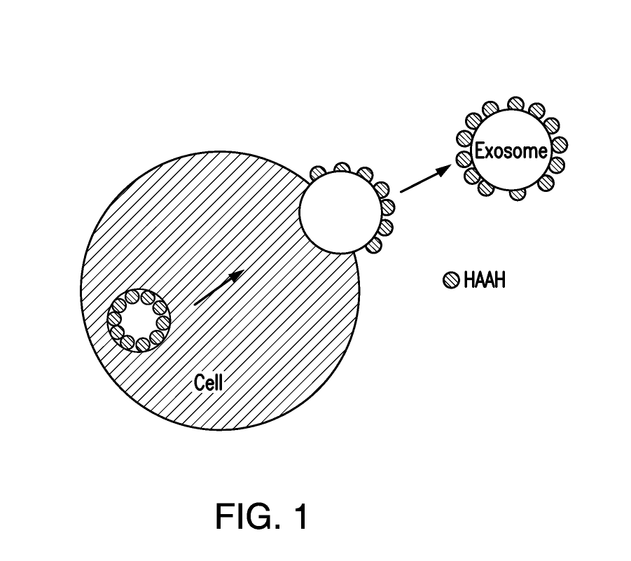

FIG. 2 depicts an exosome captured and detected with biotinylated HAAH specific antibody FB50.

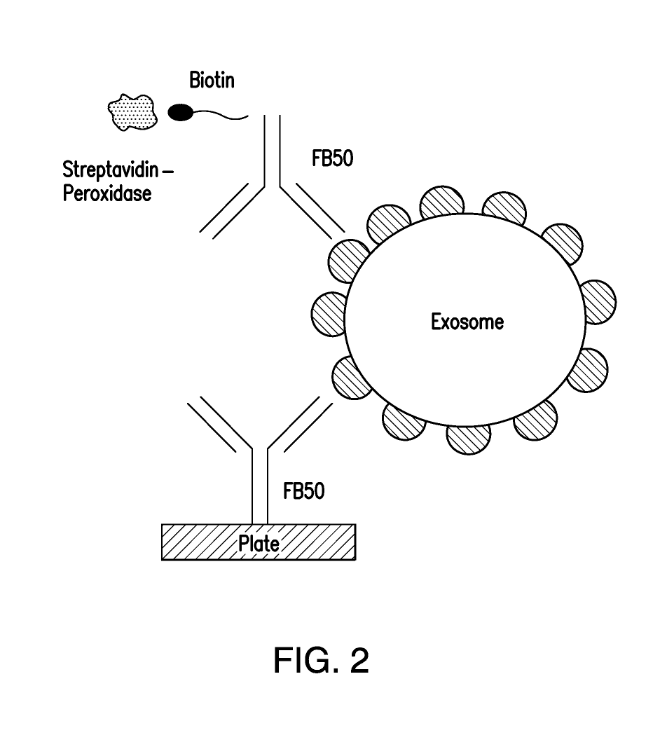

FIG. 3 depicts a typical ELISA calibration standard curve using recombinant HAAH.

FIG. 4 depicts near linearity of HAAH signal in the range of exosome sample dilution.

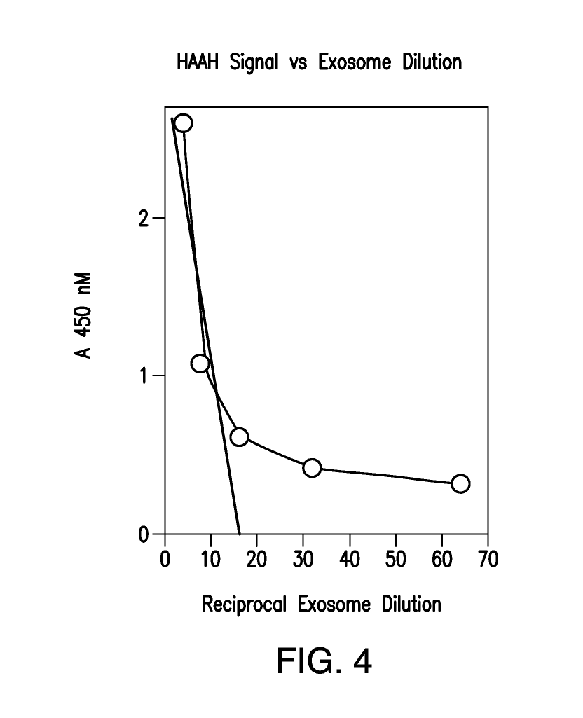

FIG. 5 depicts typical exosome particle size distribution using nanoparticle tracking analysis (NanoSight).

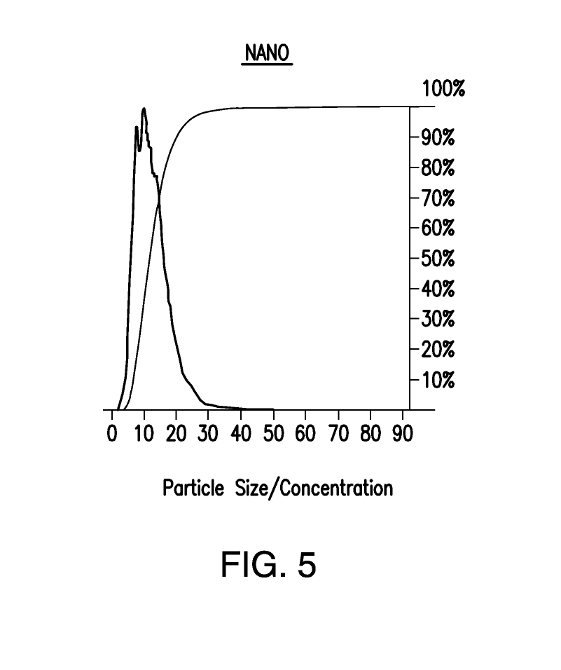

FIG. 6 shows HAAH concentrations on five different cancer patient pools and the corresponding exosome preparations of these pools.

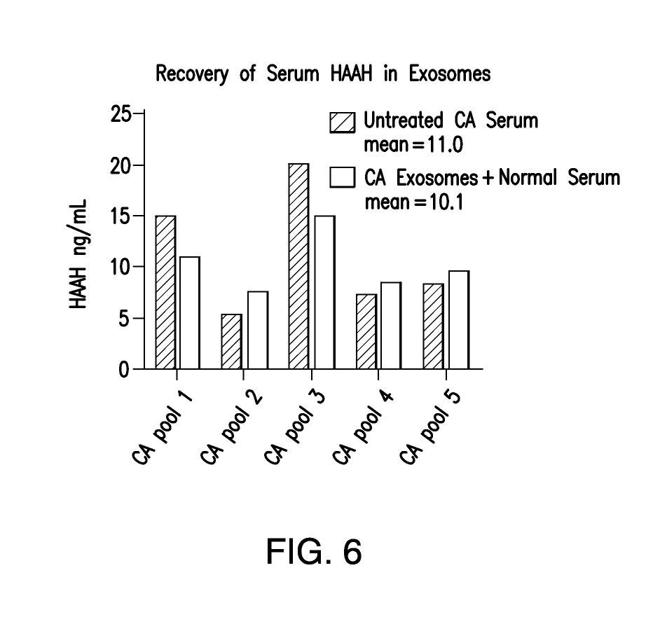

FIG. 7 shows HAAH concentrations of breast, lung, and colon cancer patients serum and in corresponding exosome preparations reconstituted with normal serum. The green dotted line represents the cutoff above which samples are regarded positive for HAAH.

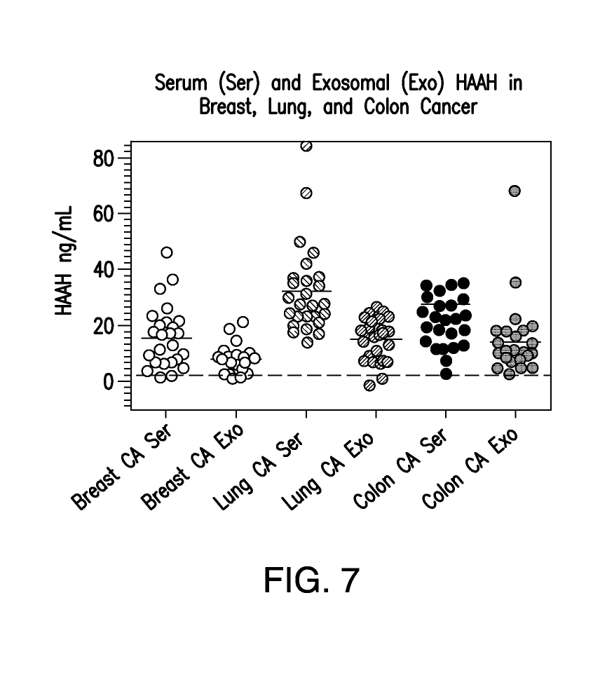

FIG. 8 shows samples from seven cancer patients that were falsely negative in the initial testing of serum. In the order indicated they were from the following cancers: prostate, breast, lung, colon, lung, bladder, and breast. The samples became positive as exosomes reconstituted with normal serum. Reconstitution with autologous serum failed to restore detection of HAAH.

FIG. 9 shows an example of an ELISA method compatible with the present invention.

### DETAILED DESCRIPTION OF THE INVENTION

For simplicity and illustrative purposes, the principles of the present invention are described by referring to various exemplary embodiments thereof. Although the preferred embodiments of the invention are particularly disclosed herein, one of ordinary skill in the art will readily recognize that the same principles are equally applicable to, and can be implemented in other systems, and that any such variation would be within such modifications that do not part from the scope of the present invention. Before explaining the disclosed embodiments of the present invention in detail, it is to be understood that the invention is not limited in its application to the details of any particular arrangement shown, since the invention is capable of other embodiments. The terminology used herein is for the purpose of description and not of limitation. Further, although certain methods are described with reference to certain steps that are presented herein in certain order, in many instances, these steps may be performed in any order as would be appreciated by one skilled in the art, and the methods are not limited to the particular arrangement of steps disclosed herein.

The accumulation of Human Aspartyl (Asparaginyl) beta Hydroxylase (HAAH) in cancer cells closely parallels significant events such as cellular differentiation, motility, and invasiveness. HAAH can be detected immunochemically as a broadly expressed once-fetal antigen both on cancer cell surfaces, and in the blood by means of an uncharacterized pathway linked to the tumor microenvironment, ectopic appearance of HAAH as a blood biomarker is now exceedingly better understood because of the recent observation that HAAH is mostly associated with exosomes. The present invention relates to HAAH immunochemical detection in the context of its physical association with the exosomal fraction of the serum matrix. By means of an HAAH selective analytical sandwich ELISA we have observed that serum HAAH is substantially associated with the exosomal fraction.

Methods for isolating exosomes are known in the art and are taught, for example, in U.S. Pat. No. 8,901,284, U.S. Pat. No. 9,005,888, and Thery et al., Cum. Protoc. Cell. Biol., Chapter 3, Unit 3: 22 (2006). Some experiments in the development of the present invention relied on ultracentrifugation of serum as a means to produce exosomes. We have also utilized commercial reagents (ExoQuick®) which permit low speed precipitation of exosomes in a micro scale high throughput fashion (See Table 1). By this method, in some cases, recovery of the HAAH antigen in exosomes prepared from the serum of cancer patients approaches 100%. This recovery while sometimes completely quantitative, on average is less and can be as low as 50%. While the nature of this sample to sample discrepancy is not understood, the results suggest that we need to focus upon optimizing the standardization of sample collection, storage, and stability of the HAAH analyte in an overall context of the exosomal matrix.

TABLE 1
Recovery of HAAH in exosomes prepared from colon cancer
serum samples using ultracentrifugation and by the commercial
exosomal precipitation reagent ExoQuick (System Biosciences).
Exosomes prepared by both methods were re-suspended in
HAAH negative normal serum before assay in the HAAH
selective ELISA.

| | HAAH ng/mL | HAAH ng/mL |
|---|---|---|
| | (exosomes- | (exosomes- |
| Sample | Exoquick) | ultracentrifugation) |
| 1 | 10.0 | 15.0 |
| 2 | 11.1 | 19.1 |
| 3 | 6.5 | 5.2 |
| 4 | 4.6 | 4.4 |
| 5 | 8.4 | 7.1 |
| 6 | 10.8 | 8.5 |
| 7 | 10.1 | 12.8 |
| 8 | 6.4 | 6.7 |
| 9 | 68.3 | 49.9 |
| 10 | 17.3 | 20.8 |
| 11 | 4.5 | 5.9 |

The present investigation has focused upon the analytical properties of HAAH recovered in the form of exosomes. Quantitative recovery of HAAH was achievable with some samples. Generally, in larger sample sets the average recovery was approximately 50%. In some samples that had a false negative determination, re-testing the exosomal fraction reconstituted with normal serum resulted in positive values. While not completely understood and the subject of ongoing investigation, there appears to be an as yet uncharacterized inhibitor in the serum of those particular samples.

### EXAMPLE 1

HAAH ELISA METHOD Using Native Serum or Serum Re-Constituted Exosomes as Test Articles

Samples for HAAH ELISA

Exosomes derived from cancer patient serum or normal volunteers were prepared either by ultracentrifugation or with the Exoquick reagent and suitably reconstituted with normal HAAH negative serum prior to use in the HAAH assay.

HAAH ELISA Calibrator

Recombinant HAAH was produced in advance of testing as an affinity purified baculovirus expressed protein and thereby served as an ELISA calibrator.

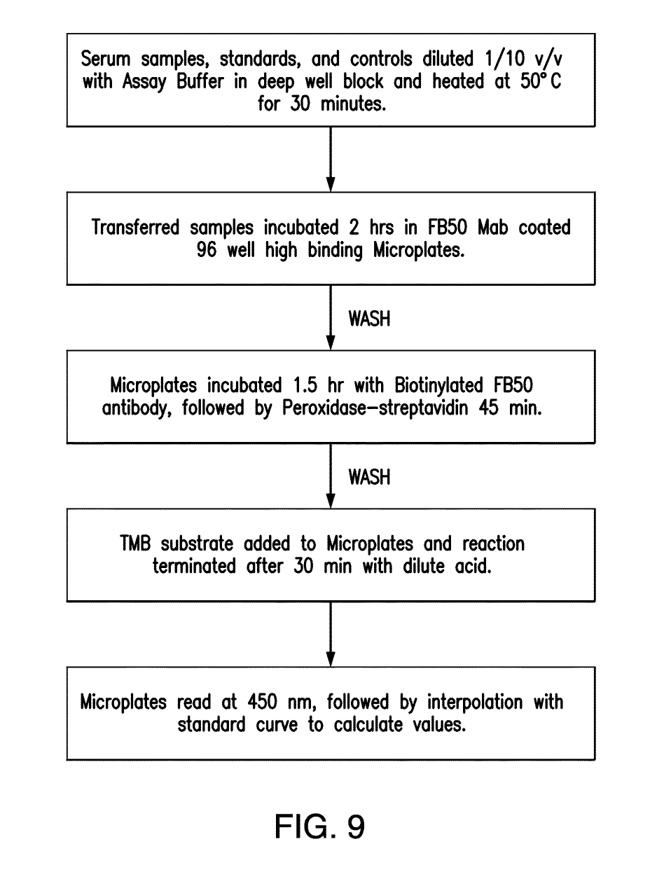

HAAH ELISA Method (see FIG. 9)

The HAAH ELISA was carried out in 96 well polystyrene microplates with monoclonal anti-HAAH FB50 in a homologous format whereby the same antibody was used for both capture and detection steps. The FB50 antibody was initially raised against the hepatoma cell line FOCUS and has been described previously in Lavaissiere, L. Jia, S. Nishiyama, M. de la Monte, S. Stern, A. M. Wands, J J. R. Friedman, P. A. (1996) J. Clin Invest. 98: 1313.

1) Serum samples, standards, and controls were first diluted 1/10 v/v with Assay buffer and subsequently heated at 50° C. for 30 minutes in a sealed polypropylene 96 well deep well plate (NUNC).

2) Treated samples were then transferred to and incubated in FB50 Mab coated/blocked high binding microplates (Costar).

3) In a sequential fashion, with intervening wash steps, the plates were then incubated with biotinylated FB50 antibody, followed by peroxidase-streptavidin (Pierce).

4) A final wash step was followed by incubation with TMB substrate (Pierce) and reaction termination with dilute acid.

5) The plates were read at 450 nm and interpolation with standard curve a used to calculate values of unknown samples.

### EXAMPLE 2

Preparation of Exosomes

Exosomes were prepared from serum by a method essentially as described by the manufacturer of the ExoQuick reagent. Serum samples and controls (40 μL) were mixed with 10 μL of ExoQuick®. After overnight incubation at 4 C the samples were centrifuged at 1500×g for 30 minutes. After aspirating the supernate the pellets were reconstituted with 40 μL pooled normal serum. Exosomes prepared in this manner were evaluated by nanoparticle tracking analysis using the NanoSight (Malvern Instruments Ltd) instrument.

The same serum samples, for comparative purposes, were suitably diluted with phosphate buffered saline (PBS) and subjected to ultracentrifugation at 100,000×g for up to 8 hours in an Optima TLX (Beckman Coulter) benchtop ultracentrifuge. After aspiration of the supernate, the exosomal pellet was resuspended in pooled normal serum.

HAAH ELISA

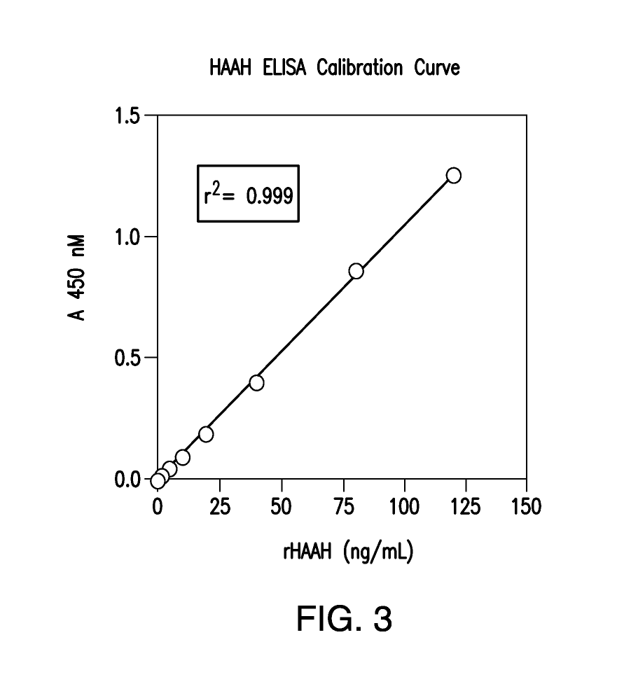

The HAAH ELISA was carried out using the same capture and detection antibody FB50 applied together in a homologous microplate format. The biotinylated FB50 detection was further amplified and readout obtained with a peroxidase/streptavidin/TMB chemistry, The assay carried out in this manner routinely yields a linear calibration standard using recombinant HAAH and has a characteristic broad dynamic range (FIG. 3). Positive and negative controls were pooled cancer patient serum and healthy donor serum respectively. A serial titration of exosomes established near linearity of signal in the working absorbance range.

Alternate Exosome Reconstitution

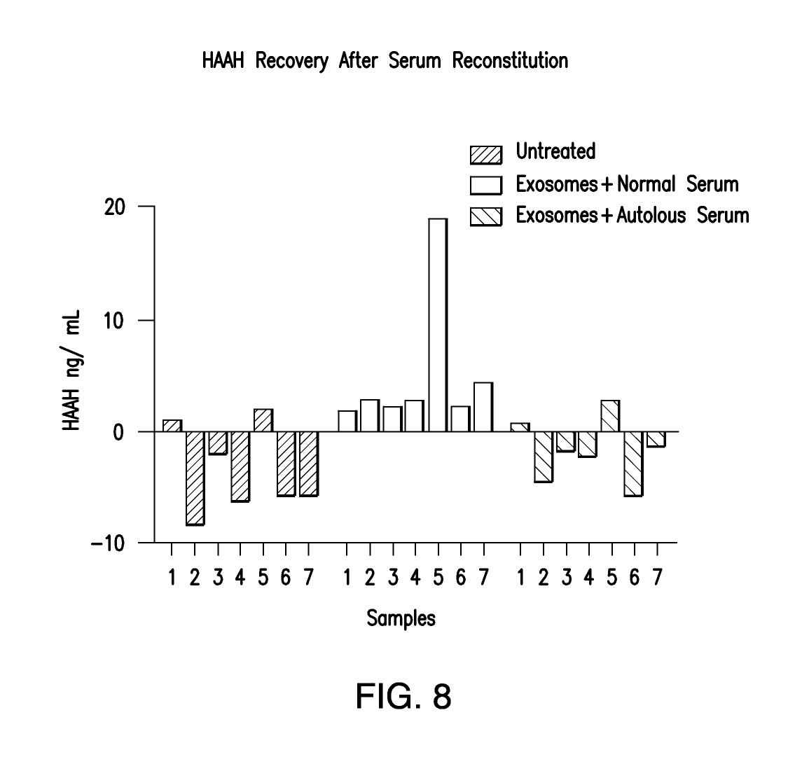

In some experiments instead of using normal serum to reconstitute exosomes, patient autologous serum was used. This was done to test for potential inhibitors of HAAH detection in false negative samples as depicted in FIG. 8.

While the invention has been described with reference to certain exemplary embodiments thereof, those skilled in the art may make various modifications to the described embodiments of the invention without departing from the scope of the invention. The terms and descriptions used herein are set forth by way of illustration only and not meant as limitations. In particular, although the present invention has been described by way of examples, a variety of compositions and processes would practice the inventive concepts described herein. Although the invention has been described and disclosed in various terms and certain embodiments, the scope of the invention is not intended to be, nor should it be deemed to be, limited thereby and such other modifications or embodiments as may be suggested by the teachings herein are particularly reserved, especially as they fall within the breadth and scope of the claims here appended. Those skilled in the art will recognize that these and other variations are possible within the scope of the invention as defined in the following claims and their equivalents.

What is claimed is:

1. A method for diagnosing cancer comprising the steps of:
isolating exosomes from a biological sample;
analyzing the exosomes for the presence of HAAH;
diagnosing cancer based on the presence of exosomes comprising HAAH.

2. The method of claim 1, wherein the exosomes are analyzed by means of ELISA.

3. The method of claim 2, wherein the ELISA is HAAH selective analytical sandwich ELISA.

4. The method of claim 1, wherein the exosomes are further analyzed for the presence of a tissue of origin specific marker selected from a group of markers but not limited to markers such as a fetoprotein, CA125, CYFRA 21-1, CEA, and PSA.

5. A method for diagnosing cancer comprising the steps of:
isolating exosomes from a sample;
resuspending exosomes isolated from the sample with magnetic beads coated with an HAAH specific antibody;
analyzing the magnetic beads for the presence of exosomes comprising HAAH.

6. The method of claim 5, wherein the exosomes comprising HAAH are captured by an HAAH specific antibody.

7. The method of claim 6, wherein the HAAH specific antibody is FB50.

8. A method for detecting the presence of HAAH in a biological sample comprising:
isolating exosomes from the biological sample;
re-suspending the exosomes isolated from the sample with magnetic beads coated with an HAAH specific antibody; and
analyzing the magnetic beads for the presence of exosomes comprising HAAH.

9. The method of claim 8, wherein the HAAH specific antibody is FB50.
